(19) United States
(12) Patent Application Publication
(10) Pub. No.: US 2009/0036800 A1
(43) Pub. Date: Feb. 5, 2009

(54) Hair Densitometer

(76) Inventors: Michael Rabin, Gates Hills, OH (US); David A. Smith, Gates Mills, OH (US); Steven Majerus, Gates Mills, OH (US)

Correspondence Address: CROCKETT & CROCKETT, P.C., 26020 ACERO, SUITE 200, MISSION VIEJO, CA, 92691, US

(21) Appl. No.: 12/182,762

(22) Filed: Jul. 30, 2008

Related U.S. Application Data
(60) Provisional application No. 60/962,705, filed on Jul. 30, 2007.

(51) Int. Cl. A61B 5/00

(52) U.S. Cl. 600/587

(57) ABSTRACT

A hair densitometer measures the number of hair shafts in a fixed area and measures the diameters of the counted hair shafts. This is done for two different areas of the head and the comparison results in a quantified measure of the thinning of a persons hair on their head.

### RELATED APPLICATIONS

This application claims priority from copending U.S. Provisional patent application 60/962,705 filed Jul. 30, 2007.

### FIELD OF THE INVENTIONS

The inventions described below relate the field of human hair growth and thinning and specifically to techniques for quantifying aspects of human hair related to pattern thinning.

### BACKGROUND OF THE INVENTIONS

Hair loss is a widespread problem in males (up to 50% affected) and a significant problem in post-menopausal women. Generally, awareness that one has a problem with hair loss occurs very late in the hair loss-process, after a significant percentage of thinning and loss has occurred.

### SUMMARY

A hair densitometer may be used to non-invasively and objectively measure multiple hair characteristics and quantify the degree of hair thinning a person is experiencing. As with most medical issues early detection is an important factor in successfully addressing the problem. Early-to-mid stage hair loss is almost entirely manifested as thinning of the hair shafts of a large percentage of follicles and retarded shaft growth so that the hairs that are present, are thin, light and close to the scalp, while follicle density and shafts per follicle are basically unchanged. Only at end-stage baldness do the follicles become irreversibly inactive. The hair densitometer may be used to measure and compare hair characteristics of one or more areas of hair from the top and or front of a human head against the same hair characteristics from one or more areas of hair from the left and or right sideburn area of a human head.

Hair thickness or density D may be quantified as the product of three factors: (1) follicular density, F, measured in follicles per square centimeter, (2) average number of hair shafts per follicle, N, and (3) average thickness, T, of the hair shafts. The product of these three quantities that indicates hair density D.

D=F*N*T

Another possible objective measure of hair thinning may be the fraction of hair widths outside two standard deviations for a selected subset of hair from the head.

In another alternative measure of hair density, hair thickness (diameter) may be replaced with hair cross sectional area A=πT²/4. The overall sense or indicator of “fullness” of a head of hair may also include the hair length L, resulting in a hair volume parameter V=F*N*A*L. Since one can increase the sense of hair volume by letting remaining hair grow, V serves as an appearance metric but masks hair thinning. Thus, D=FNA will be the objective standard used.

Typical follicular unit densities are in the range of 60-120 cm⁻² and each follicle generally contains one or two shafts, but rarely, more than two hair shafts of varying ages. Hair shaft thickness may be classified as coarse, medium or fine and the mean value of the shaft thickness will vary from about 40 microns in width for fine hair, while coarse hair might average 90 microns in width. N will generally be a number between 1 and 2, and more commonly 1-1.25 it can be eliminated from the density determination but may need to be considered in some rare cases.

A normalized hair density measurement for every individual is the ratio of top and or front hair density to left and or right side hair density. Thus, an individual's hair thinning ratio may be expressed as:

R=100×(Dₜₒₚ)/D_{side}

With Dₜₒₚ and D_{side}=F*N*T for each respective region.

A hair densitometer may employ optical and or electronic techniques combined with mechanical manipulation to obtain an objective measure of hair thinning on a human head. The mechanical system nondestructively engages, separates and aligns hair to be analyzed. Then a scanning magnification system illuminates a linear detector array to automatically measure, record and analyze hair widths. One measure of thinning is the ratio R determined between a test area and a reference area. Another measure of hair density relates to the fraction of hairs that are more than a certain number N of standard deviations below the mean measured thickness will be defined as one potential hair loss and or thinning factor. Yet another assessment of hair thinning may be determined from the change in dielectric constant of an aggregation of hair shafts in a test and a control area of a scalp.

### BRIEF DESCRIPTION OF THE DRAWINGS

FIG. 1 is a graph of a hair width spectrum.

FIG. 2 is a graph of hair width spectrums taken from different areas of a human head.

FIG. 3 is a diagram of a hair sample technique taken along a part line.

FIG. 4 is a comparison diagram of actual hair shafts at two locations in a target region relative to the detector output.

FIG. 5 is an exploded view of a scanning system for a hair densitometer.

FIG. 6 is a set of comparison views of hair scans from different areas of a human head and a calibration scale.

FIG. 7 is photograph of a hair densitometer applied to a head.

FIG. 8 is several perspectives of a hair densitometer.

FIG. 9 is a table of hair density analysis for different areas of a head.

FIG. 10 is a block diagram of an optical detector for a hair densitometer.

FIG. 11 is a diagram of a detector output signal related to a hair sample viewed by the optical detector.

FIG. 12 is a perspective view of a capacitive comb for hair density analysis.

FIG. 13 is a perspective view of a technique for capacitive analysis of hair density.

### DETAILED DESCRIPTION OF THE INVENTIONS

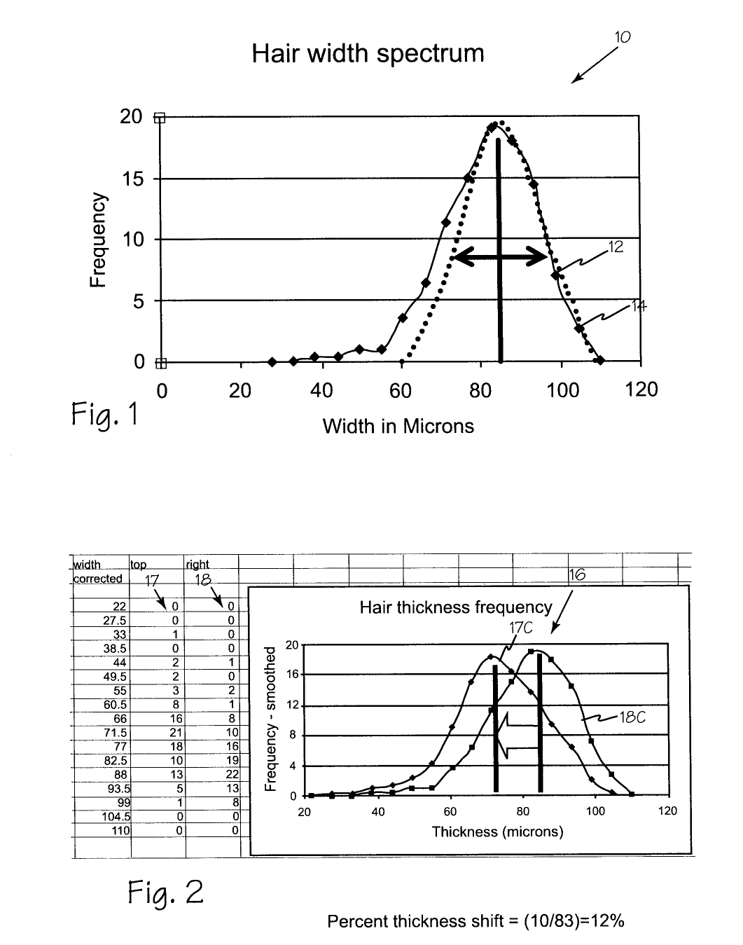

In FIG. 1, graph 10 represents hair thickness measurements for a sample of 100 hairs taken from the top of the head of a 58-year old with a receding hairline. Thickness measurements were obtained by aligning hairs on a millimeter scale and capturing a digital microscope image under various magnifications against a 1 mm registration scale. The width of each hair shaft was measured to 0.5 mm accuracy on ten blown-up images which were scaled according to the 1 mm registration lines. Data points such as data point 12 were smoothed by replacing the data count with the average of the data count for data point 12 and the data count for its nearest neighbors such as data point 14.

Graph 16 of FIG. 2 illustrates the difference between hair samples from the top of a head such as data 17 and curve 17C, with samples from the side of the same head such as data 18 and curve 18C.

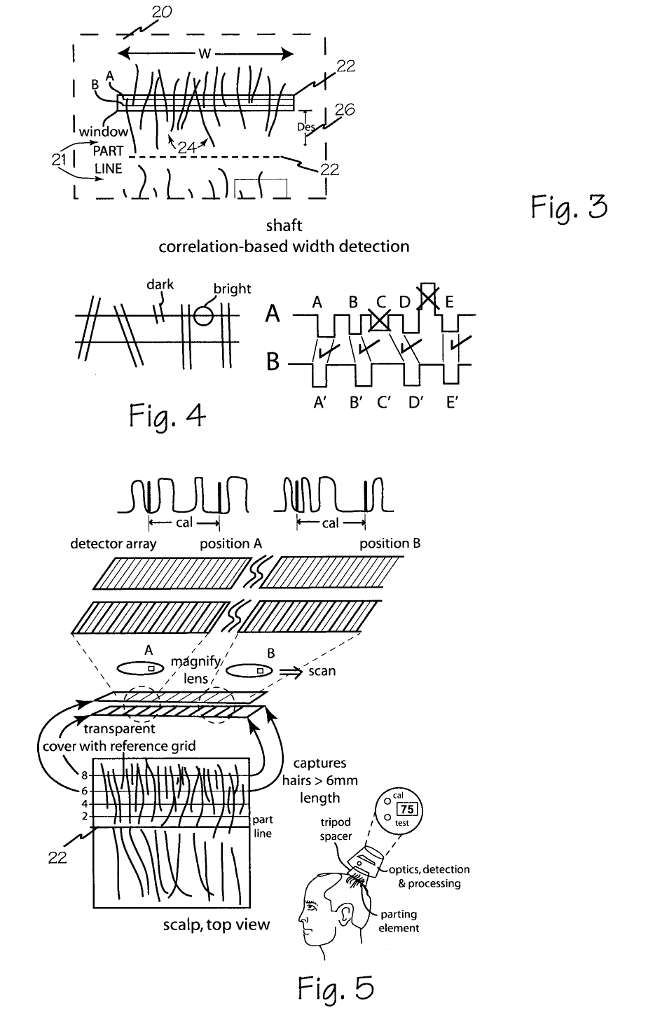

A hair densitometer technique illustrated in FIGS. 3, 4 and 5 includes identifying two different target regions such as target region 20 on a subjects head. For example, target regions might include top and right side, front and right side, top and left side or front and left side. Hair 21 in each target region is parted along a part line 22 forming a test region 23. A hair densitometer measures the count and thickness of hairs 24 at a calibrated distance 26 above the part line in the test region.

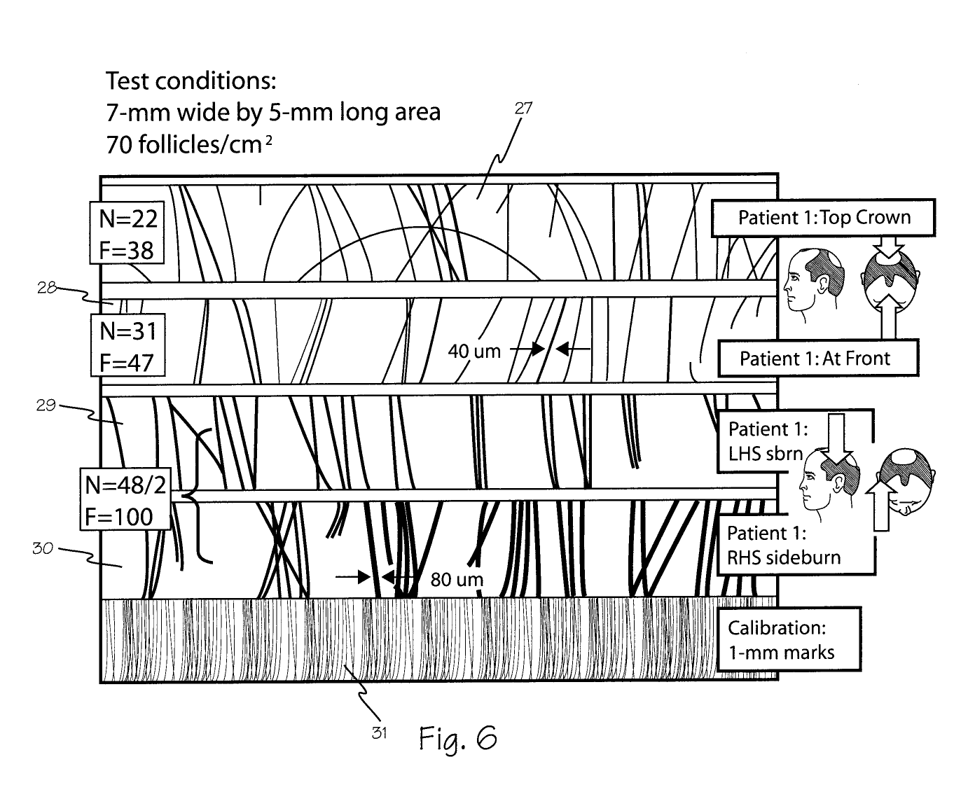

Referring now to FIG. 6, test regions 27, 28, 29 and 30 illustrate varying hair characteristics as a function of location on a head. Calibration scale 31 is available to enable accurate determination of hair shaft diameter.

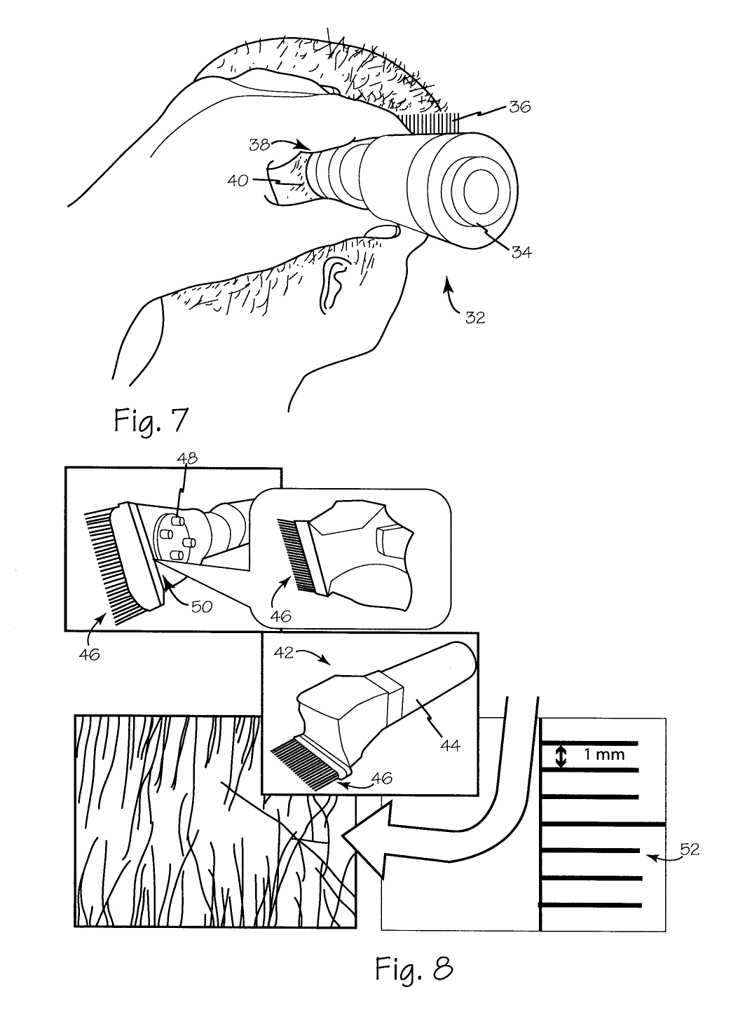

Hair Densitometer 32 of FIG. 7 includes a magnifying imaging apparatus 34 and any suitable mechanical apparatus to align hair for imaging such as comb attachment 36 and any suitable illumination source. Hair analysis images are obtained through a window such as window 38 that includes a calibration scale such as mm scale 40.

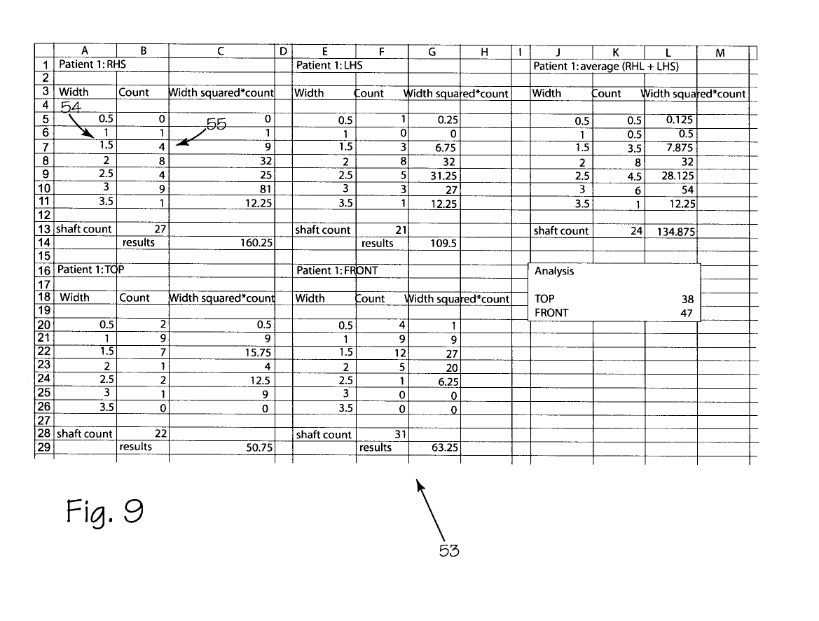

Alternate Hair Densitometer 42 of FIG. 8 includes a magnifying imaging apparatus 44 and any suitable mechanical apparatus to align hair for imaging such as comb attachment 46 and any suitable illumination source such as LEDs 48. Hair analysis images are obtained through a window such as window 50 that includes a calibration scale such as mm scale 52. Hair visible through window 50 may be counted and measured by a technician or by any suitable automated system. When the hair is counted and measured by a human, data table 53 as illustrated in FIG. 9 may be created to compile data such as measurement data 54 and count data 55.

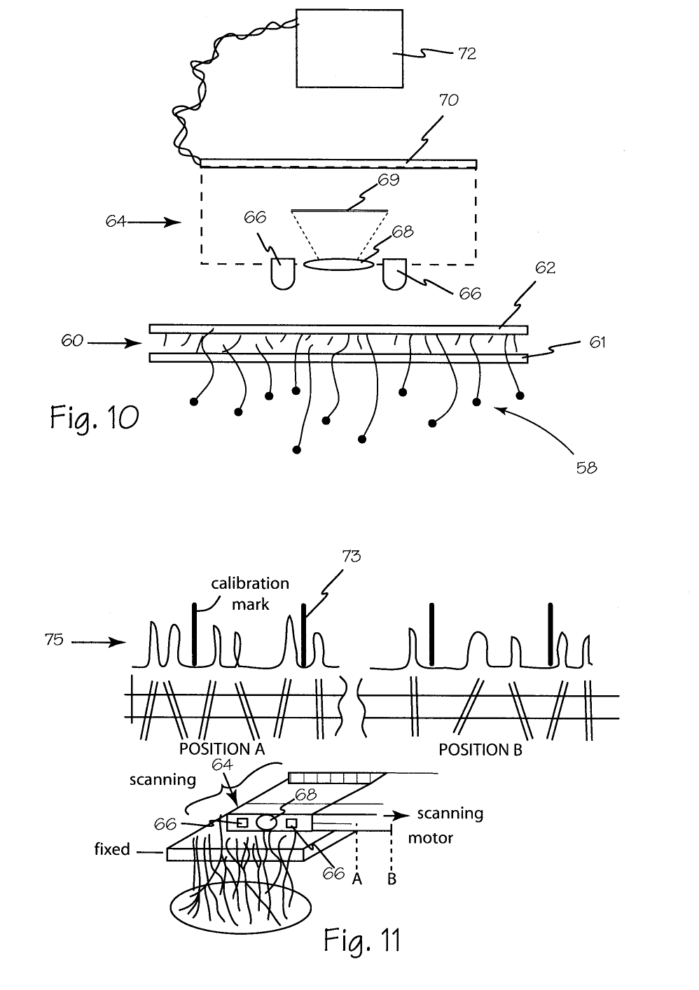

Automated hair densitometer 56 of FIG. 10 engages hair 58 from a test area of a subject's head into collector area 60 where hair 58 is pressed between a suitably colored background 61, here white, and clear plate 62. Scanning assembly 64 includes LEDs 66 and magnifying lens 68 which magnifies the view of collector area 60 and projects image 69 onto detector 70. Scanning assembly may also include incremental scanning of collector area 60 into for example, 2-4 mm portions as shown in FIG. 11. Upon completion of scan data capture, the data may be processed by processor 72 as discussed above. Calibration scale 73 may be identified in detector signal 75.

The performance of a hair densitometer as described may be altered by the use of alternative illumination techniques such as polarized light, multiple wavelength light sources, infrared or UV sources. Imaging and or processing improvements for grey, blonde and or other light colored hair types may also be employed such as for example, washable dyes or coatings, scalp coloring or other suitable techniques.

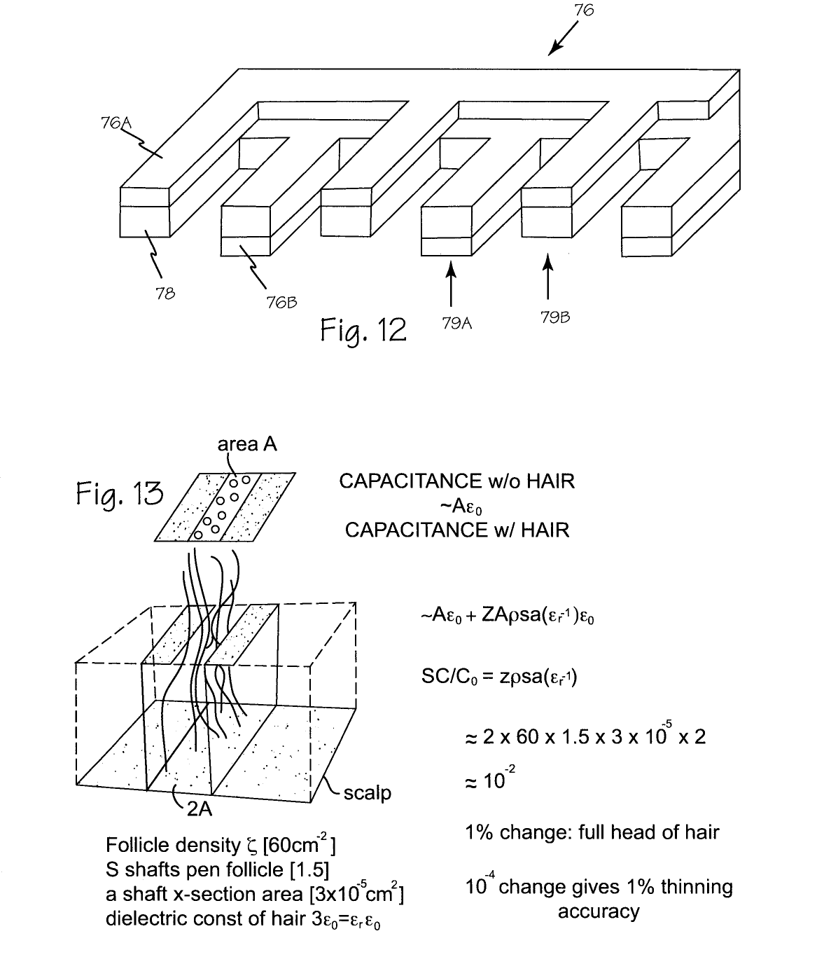

Referring now to FIG. 12 and FIG. 13, an alternate hair densitometer may adapt capacitive techniques to replace imaging in other configurations. Capacitive comb 76 includes capacitive plates 76A and 76B separated by insulating layer 78. Any hair shafts engaged between comb fingers such as fingers 79 and 80 will alter the capacitance and thus may be detectable and quantifiable. Because human hair has a different dielectric constant than air, the insertion of just a single hair in the comb finger capacitor will cause a noticeable change in capacitance. This change can be integrated over a long period of time, thereby constituting a huge over-sampling ratio. This integration period will be chosen to be long enough to negate the effects of phase-noise on non-ideal resonant networks. Essentially, this method allows for cheap but accurate transduction of low-frequency signals. Any observed capacitance shifts will occur due to hair density variations.

By placing the comb-capacitor in a resonant network such as a free-running oscillator a non-imaging detector may be formed. In this topology, any capacitance changes in the comb structure will correspond to linear changes in the oscillation frequency of the circuit. A microcontroller can count the number of cycles per second to obtain a rough readout of the capacitance value. Thus if two measurements are performed and the number of cycles per second are significantly different, then the hair densities are quite different.

For instance, suppose that the free-running frequency of the oscillator in air is 100 kHz. As soon as any non-conductive material (such as hair) passes between the comb fingers, the capacitance of the structure will increase. This increase in capacitance will reduce the 100 kHz free-running frequency.

Insertion of the comb into a relatively dense section of hair (side-of-head) will reduce this frequency of oscillation by as much as a one to a few percent. Integrated over a long sample-time of one second, this corresponds to a change of a few thousand digitally-detectable cycles. When the comb is applied to a less-dense area of the scalp (for instance the top), the oscillation frequency will approach the free-running frequency. A simple measure of hair density is therefore proportional to the difference in the two measured frequencies. In short, the magnitude of the difference in oscillation frequencies will correspond to the magnitude in the difference in hair densities.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A hair densitometer comprising:
means for engaging a hair sample;
a detector for counting each hair shaft in a hair sample and providing a count signal;
a hair measurement comparison standard;
a measurement device for measuring the diameter of each hair shaft in a hair sample, comparing each hair shaft diameter against the hair measurement comparison standard and providing hair diameter signals;
means for saving the count signal and the hair diameter signals;
means for comparing the saved count signals and diameter signals from a first hair sample and a second hair sample to generate a hair density signal, the first and second hair samples taken at different areas of a human head.

2. A method of measuring hair density comprising the steps:
selecting a first target area on the top of a person's head;
counting the number of hair shafts in the target area to generate first count data;
measuring the diameter of each counted hair shaft to generate first measurement data;
processing the first count and first measurement data to determine a hair density for the first target area;
selecting a second target area on the side of a person's head;
counting the number of hair shafts in the second target area to generate second count data;
measuring the diameter of each counted hair shaft to generate second measurement data;
processing the second count and second measurement data to determine a hair density for the second target area;
comparing the hair density for the first target area to the hair density for the second target area to generate a hair density measurement.

3. A hair densitometer comprising:
means for engaging a hair sample having a dielectric constant corresponding to the number and diameter of hair shafts engaged;
a detector connected to the engaging means, the detector using the dielectric constant of the engaging means to generate a density signal;
means for saving the density signal;
means for comparing the density signals from a first hair sample and a second hair sample to generate a hair density signal, the first and second hair samples taken at different areas of a human head.
